(19) United States
(12) Patent Application Publication
(10) Pub. No.: US 2010/0035904 A1
(43) Pub. Date: Feb. 11, 2010

(54) PHENOBARBITAL SALTS; METHODS OF MAKING; AND METHODS OF USE THEREOF

(76) Inventors: Tong Sun, Marlton, NJ (US); Shawn Watson, Cherry Hill, NJ (US); Rajesh Manchanda, Morton, PA (US)

Correspondence Address: CANTOR COLBURN, LLP, 20 Church Street, 22nd Floor, Hartford, CT, 06103, US

(21) Appl. No.: 12/537,304

(22) Filed: Aug. 7, 2009

Related U.S. Application Data
(60) Provisional application No. 61/087,348, filed on Aug. 8, 2008.

(51) Int. Cl. A61K 31/515; C07D 239/02

(52) U.S. Cl. 514/270; 544/304

(57) ABSTRACT

Disclosed are new phenobarbital salts, methods of preparation, and uses thereof.

### CROSS REFERENCE TO RELATED APPLICATION

This application claims the benefit of U.S. Provisional Application Ser. No. 61/087,348 filed Aug. 8, 2008, which is hereby incorporated by reference in its entirety.

### BACKGROUND

Phenobarbital, 5-ethyl-5-phenyl-2,4,6(1H,3H,5H)-pyrimidinetrione, is a well-known anticonvulsant, sedative, and hypnotic. The free acid form of phenobarbital has a water solubility of one gram in about one liter of water.

One approach to forming a more soluble version of an active agent is to form a salt. Use of a particular salt form may provide improved solubility, dissolution, and possibly increased bioavailability over the free form of an active agent.

Phenobarbital sodium is a known salt freely soluble in water in contrast to the free acid form. However, phenobarbital sodium is hygroscopic and degrades when exposed to humid environments.

There remains a need in the art for new salt forms of phenobarbital having increased aqueous solubility. There also remains a need in the art for new salt forms of phenobarbital having improved properties of increased aqueous solubility while at the same time exhibiting chemical stability.

### SUMMARY

In one embodiment, a crystalline salt of phenobarbital is selected from the group consisting of phenobarbital potassium, phenobarbital benzathine, phenobarbital betaine, phenobarbital choline, and corresponding anhydrous forms, hydrates, solvates, or polymorphic forms thereof.

In another embodiment, a method of preparing a crystalline salt of phenobarbital comprises crystallizing or precipitating a phenobarbital salt from a solution of phenobarbital, a counterion source, and a solvent.

In yet another embodiment, a composition comprises a compound which is a crystalline salt of phenobarbital selected from the group consisting of phenobarbital potassium, phenobarbital benzathine, phenobarbital betaine, phenobarbital choline, and corresponding anhydrous forms, hydrates, solvates, or polymorphic forms thereof; and a pharmaceutically acceptable excipient.

In still another embodiment, a method of treating comprises administering to a patient in need of phenobarbital therapy a compound which is a crystalline salt of phenobarbital selected from the group consisting of phenobarbital potassium, phenobarbital benzathine, phenobarbital betaine, phenobarbital choline, and corresponding anhydrous forms, hydrates, solvates, or polymorphic forms thereof; or a composition comprising the crystalline salt and a pharmaceutically acceptable excipient.

These and other embodiments, advantages and features of the present invention become clear when detailed description and examples are provided in subsequent sections.

### DETAILED DESCRIPTION

Disclosed herein are new crystalline salts of phenobarbital including phenobarbital potassium, phenobarbital benzathine, phenobarbital betaine, phenobarbital choline, and corresponding anhydrous forms, hydrates, solvates, or polymorphic forms thereof. Also disclosed herein are non-crystalline or amorphous forms of phenobarbital salts including phenobarbital sodium, phenobarbital potassium, phenobarbital benzathine, phenobarbital betaine, phenobarbital choline, and the corresponding anhydrous forms, hydrates, or solvates thereof.

The new salts of phenobarbital all exhibit increased water solubility over the free acid form. The water solubility of phenobarbital potassium is about 88 milligrams per milliliter (mg/ml), phenobarbital benzathine is about 2.9 mg/ml, phenobarbital betaine is about 1.9 mg/ml, and phenobarbital choline is about 295 mg/ml.

Furthermore, salt forms such as phenobarbital benzathine is nonhygroscopic as compared to known phenobarbital sodium.

The phenobarbital salts can be prepared by making a solution containing phenobarbital, the desired source of counter ion (e.g., potassium hydroxide, choline, betaine, or benzathine) and a solvent, optionally with the addition of an anti-solvent, and allowing the crystals of the phenobarbital salts to form. The temperature of the crystallization solution can initially be high followed by gradual reduction in temperature to initiate crystal formation. Alternatively, the solvent of the crystallization solution can slowly be evaporated.

In one embodiment, the crystallization solution, prior to any solids formation, can be filtered to remove any undissolved solids, solid impurities and the like prior to removal of the solvent. Any filtration system and filtration techniques known in the art can be used.

In one embodiment, the crystallization solutions can be seeded with the desired crystalline phenobarbital salt.

In an alternative embodiment, the phenobarbital salt can be formed by a precipitation method involving removing the solvent from a solution of containing phenobarbital, the desired source of counter ion (e.g., potassium hydroxide, choline, betaine, or benzathine) and a solvent.

Suitable solvents for preparing the phenobarbital salts include those that do not adversely affect the stability of the salt, and are preferably inert. Suitable solvents may be organic, aqueous, or a mixture thereof Suitable organic solvents may be aliphatic alcohols such as methanol (MeOH), ethanol (EtOH), n-propanol, isopropanol (IPA), n-butanol, tert-amyl alcohol (t-AmOH); ethers such as tetrahydrofuran (THF), dioxane, methyl-tert-butyl ether, 1,2-dimethoxyethane (DME), and 2-methyl tetrahydrofuran; aliphatic ketones such as acetone, methyl ethyl ketone (MEK), and methyl isobutyl ketone; aliphatic carboxylic esters such as methyl acetate, ethyl acetate (EtOAc), and isopropyl acetate; aromatic hydrocarbons such as benzene, toluene, and xylene; aliphatic hydrocarbons such as hexane; aliphatic nitriles such as acetonitrile (MeCN); chlorinated hydrocarbons such as dichloromethane (DCM), chloroform, and carbon tetrachloride; aliphatic sulfoxides such as dimethyl sulfoxide (DMSO); amides such as dimethylformamide (DMF) and dimethylacetamide (DMA); organic acids such as acetic acid; N-methyl-2-pyrrolidone; pyridine; and the like, as well as mixtures comprising at least one of the foregoing organic solvents. Other solvents can be used as an anti-solvent to induce crystal formation of the phenobarbital salt from solution. Exemplary anti-solvents include those solvents for which the phenobarbital salt is not readily soluble in.

In one embodiment, phenobarbital potassium is prepared by precipitation from a solution of phenobarbital, potassium hydroxide, and isopropanol via removal of the isopropanol.

In another embodiment, phenobarbital betaine is prepared by precipitation from a solution of phenobarbital, betaine, and dimethylformamide via removal of the dimethylformamide.

In one embodiment, phenobarbital choline is prepared by precipitation from a solution of phenobarbital, choline, and isopropanol via removal of the isopropanol.

In yet another embodiment, phenobarbital benzathine is prepared by precipitation from a solution of phenobarbital, benzathine, and tetrahydrofuran via removal of the tetrahydrofuran.

Also disclosed herein are pharmaceutical compositions comprising the phenobarbital salts prepared herein.

Solid dosage forms for oral administration include, but are not limited to, capsules, tablets, powders, and granules. In such solid dosage forms, the phenobarbital salt may be admixed with one or more of the following: (a) one or more inert excipients (or carriers), such as sodium citrate or dicalcium phosphate; (b) fillers or extenders, such as microcrystalline cellulose, starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as sodium starch glycolate, sodium or calcium carboxymethylcellulose, croscarmellose sodium, crospovidone, pregelatinized starch, agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, (k) buffering agents, (l) glidants such as silicon dioxide, and combinations comprising one or more of the foregoing additives.

A “dosage form” means a unit of administration of an active agent. Examples of dosage forms include solid dosage forms such as tablets, capsules, and films, liquid dosage forms such as liquids, solutions, and suspensions, injections, emulsions, creams, ointments, suppositories, inhalable forms, transdermal forms, and the like. The dosage form may be administered via oral, buccal, parenteral, pulmonary, or transdermal administration. Parenteral administration includes intravenous, intramuscular, subcutaneous (which may be in the form of a subcutaneous depot), and the like.

By “oral dosage form” is meant to include a unit dosage form for oral administration. An oral dosage form may optionally comprise a plurality of subunits such as, for example, microcapsules or microtablets. Multiple subunits may be packaged for administration in a single dose, such as minitablets in a capsule.

By “subunit” is meant to include a composition, mixture, particle, pellet, etc., that can provide an oral dosage form alone or when combined with other subunits. Exemplary subunits include minitablets, beads, spheroids, microspheres, seeds, pellets, caplets, microcapsules, and granules.

The compositions can be immediate-release forms or controlled-release forms.

By “immediate-release” is meant a conventional or non-modified release in which greater then or equal to about 75% of the active agent is released within two hours of administration, specifically within one hour of administration.

By “controlled-release” is meant a dosage form in which the release of the active agent is controlled or modified over a period of time. Controlled can mean, for example, sustained-, delayed- or pulsed-release at a particular time. Alternatively, controlled can mean that the release of the active agent is extended for longer than it would be in an immediate-release dosage form, e.g., at least over several hours.

“Sustained-release” or “extended-release” include the release of the active agent at such a rate that blood (e.g., plasma) levels are maintained within a therapeutic range for at least about 8 hours, specifically at least about 12 hours, and more specifically at least about 24 hours after administration at steady-state. The term steady-state means that a plasma level for a given active agent has been achieved and which is maintained with subsequent doses of the drug at a level which is at or above the minimum effective therapeutic level for a given active agent.

By “delayed-release”, it is meant that there is a time-delay before significant plasma levels of the active agent are achieved. A delayed-release formulation of the active agent can avoid an initial burst of the active agent, or can be formulated so that release of the active agent in the stomach is avoided and absorption occurrs in the small intestine.

Certain compositions described herein may be “coated”. The coating may be a suitable coating, such as, a functional or a non-functional coating, or multiple functional or non-functional coatings. By “functional coating” is meant to include a coating that modifies the release properties of the total composition, for example, a sustained-release coating. By “non-functional coating” is meant to include a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition.

Dosage forms can be combination dosage forms having both immediate-release and controlled-release characteristics, for example, a combination of immediate-release subunits and controlled-release subunits, either extended-release or delayed-release. The combination of immediate-release and delayed-release subunits can provide a pulsed-release profile. The immediate-release portion of a combination dosage form may be referred to as a loading dose. Still other embodiments include two, three, or more types of controlled-release subunits differing in their respective release profiles. The controlled-release subunits may differ in the composition of a release-retarding matrix or coating; or differ in quantity of the same type of release-retarding matrix or coating material.

Subunits may be prepared by, for example, dry granulation or wet granulation followed by compression or compaction, melt extrusion and spheronization, layering (e.g., spray layering suspension or solution), and the like. Examples of such techniques include direct compression, using appropriate punches and dies, the punches and dies are fitted to a suitable rotary tableting press; injection or compression molding using suitable molds fitted to a compression unit, granulation followed by compression; and extrusion in the form of a paste, into a mold or to an extrudate to be cut into lengths.

The controlled-release subunits may be prepared using release-retarding matrix material or a controlled-release coating. In one embodiment, both a release-retarding matrix material and a controlled-release coating are used. Exemplary release-retarding matrix materials include a poly(meth)acrylate, an alkylcellulose, shellac, zein, a wax, a hydrogenated vegetable oil, a hydrogenated castor oil, polyvinylpyrrolidine, a vinyl acetate copolymer, a polyethylene oxide, and the like, or a combination comprising at least one of the foregoing materials.

The subunits may be coated with a controlled-release coating comprising a release-retarding material and optional excipients such as plasticizers, pore-forming component, surfactants, and the like. Exemplary release retarding materials for the coating include chitosan, ethylcellulose, (e.g. ethylcellulose, such as AQUACOAT, a 30% dispersion available from FMC, Philadelphia, Pa.; SURELEASE a 25% dispersion available from Colorcon, West Point, Pa.; Ethocel; or Aqualon); hydroxypropyl methylcellulose acetate succinate (HPMCAS); cellulose acetate phthalate (CAP); a (meth)acrylic acid copolymer; hydroxypropyl methylcellulose succinate; cellulose acetate succinate; cellulose acetate hexahydrophthalate; hydroxypropyl methylcellulose hexahydrophthalate; hydroxypropyl methylcellulose phthalate (HPMCP); cellulose propionate phthalate; cellulose acetate maleate; cellulose acetate trimellitate; cellulose acetate butyrate; cellulose acetate propionate; a poly(meth)acrylic acid; a poly(meth)acrylate; a polyvinylacetate phthalate; zein; and the like, or a combination comprising at least one of the foregoing materials. “(Meth)acrylic or (meth)acrylate” is inclusive of acrylic, methacrylic, acrylate, or methacrylate.

Exemplary polymethacrylates include copolymers of acrylic and methacrylic acid esters, such as a. an aminomethacrylate copolymer USP/NF such as a poly(butyl methacrylate, (2-dimethyl aminoethyl)methacrylate, methyl methacrylate) 1:2:1 (e.g., EUDRAGIT E 100, EUDRAGIT EPO, and EUDRAGIT E 12.5; CAS No. 24938-16-7); b. a poly(methacrylic acid, ethyl acrylate) 1:1 (e.g., EUDRAGIT L30 D-55, EUDRAGIT L100-55, EASTACRYL 30D, KOLLICOAT MAE 30D AND 30DP; CAS No. 25212-88-8); c. a poly(methacrylic acid, methyl methacrylate) 1:1 (e.g., EUDRAGIT L 100, EUDRAGIT L 12.5 and 12.5 P; also known as methacrylic acid copolymer, type A NF; CAS No. 25806-15-1); d. a poly(methacrylic acid, methyl methacrylate) 1:2 (e.g. EUDRAGIT S 100, EUDRAGIT S 12.5 and 12.5P; CAS No. 25086-15-1); e. a poly(ethyl acrylate, methylmethacrylate, trimethylammonioethyl methacrylate chloride) 1:2:0.2 or 1:2:0.1 (e.g., EUDRAGITS RL 100, RL PO, RL 30 D, RL 12.5, RS 100, RS PO, RS 30 D, or RS 12.5; CAS No. 33434-24-1); f. a poly(ethyl acrylate, methyl methacrylate) 2:1 (e.g. EUDRAGIT NE 30 D; CAS No. 9010-88-2); and the like, or a combination comprising at least one of the foregoing materials.

In one embodiment, the subunit, such as a minitablet, has an average diameter of about 50 micrometers to about 5000 micrometers, specifically about 100 to about 3000, more specifically about 150 to about 2000, still yet more specifically about 250 to about 1500, and yet more specifically about 500 to about 1000 micrometers. These diameters are inclusive of either uncoated or coated subunits.

In certain embodiments, an optional intermediate subcoating is used between the subunit core and the coating providing controlled-release properties. Such an intermediate coating can be used to protect the active agent or other component of the core subunit from the material used in the controlled-release coating. Exemplary intermediate coatings include film forming polymers such as hydroxyethyl cellulose, hydroxypropyl cellulose, gelatin, hydroxypropyl methylcellulose, polyethylene glycol, polyethylene oxide, and the like, or a combination comprising at least one of the foregoing; and a plasticizer.

The dosage forms may contain a therapeutic amount of phenobarbital salt. Exemplary amounts of the phenobarbital salt in the dosage form can be about 1 to about 400 mg, specifically about 20 to about 300 mg, more specifically about 50 to about 100 mg of phenobarbital potassium, phenobarbital benzathine, phenobarbital betaine, phenobarbital choline, or the corresponding anhydrous forms, hydrates, solvates, or polymorphic forms thereof.

In one embodiment, the composition comprising the phenobarbital salt is a parenteral dosage form, either prepared as a ready-to-use solution or suspension, or as a dry solid (e.g., lyophilizate) which can be mixed with a liquid vehicle prior to use. The advantage of using the phenobarbital salt for a parenteral is its increased solubility, particularly in water, as compare to the solubility of free phenobarbital. Further, increased solubility can result in reduced amounts of liquid vehicle needed to prepare the parenteral, thereby resulting in lower quantities of material to inject into the patient. Such benefits can be significant, particularly for the treatment of neonatal seizures. The parenteral solution generally can contain the phenobarbital salt and a pharmaceutically acceptable parenteral excipient, for example a parenteral vehicle (aqueous or nonaqueous); an antimicrobial; an antioxidant; a chelating agent; a buffer; a tonicity adjustment agent; and the like; and combinations thereof. Exemplary parenteral vehicles include water, ethanol, polyethylene glycol, propylene glycol, fixed oils, and the like, and combinations thereof. Exemplary antimicrobial agents include benzyl alcohol, parabens (e.g., methyl paraben, propyl paraben), benzethonium chloride, phenols, and the like, and combinations thereof.

In another embodiment, the composition comprising the phenobarbital salt is an oral, liquid dosage form, either prepared as a ready-to-use solution or suspension, or as a dry solid which can be mixed with a liquid vehicle prior to use. Free phenobarbital has limited solubility in water, thus the use of the phenobarbital salts which have increased solubility can be used to prepare liquid solutions or suspensions. The oral, liquid dosage form generally can contain the phenobarbital salt and a pharmaceutically acceptable excipient, for example a liquid vehicle (aqueous or nonaqueous); an antimicrobial; an antioxidant; a chelating agent; a buffer; an emulsifier; a sweetening agent; a flavor; a color; and the like; and combinations thereof. Exemplary liquid vehicles include water, ethanol, glycerin, propylene glycol, polyethylene glycol, and the like, and combinations thereof. Exemplary sweeteners include sucrose, sugar alcohols (e.g., sorbitol, mannitol), artificial sweeteners, and the like, and combinations thereof.

The phenobarbital salts and compositions prepared therefrom can be used as an antiepileptic/anticonvulsant, a sedative, or a hypnotic. The salts and compositions are useful in treating or controlling conditions such as seizures, including grand mal seizures, complex or simple partial seizures in adults and children, generalized tonic-clonic seizures, myclonic seizures and neonatal seizures, status epilepticus; short-term treatment of anxiety; or short-term treatment of insomnia.

The phenobarbital salts can be used alone or in combination with other antiepiletic agents (e.g., carbamazepine, diphenylhydantoin (“phenytoin”), lamotrigine, pentobarbital, primidon, valproate, valproic acid, etc.); bronchodilators; vasodilators (e.g., amlodipine, felodipine, nicardipine, nifedipine, etc.); analgesics; or anticholenergics.

The following examples further illustrate the invention but, of course, should not be construed as in any way limiting its scope.

### EXAMPLES

### Experimental Conditions

Moisture sorption experiments are carried out on about 10 milligrams (mg) of sample in a Pyrex bulb by first equilibrating the sample at 25° C./40% relative humidity (RH) until an equilibrium weight is reached. The sample is then subjected to an isothermal (25° C.) adsorption scan from 40 to 90% RH in steps of 10% RH. The sample is allowed to equilibrate to either an asymptotic weight or a maximum of four hours for each relative humidity. A desorption scan from 90 to 0% RH (at 25° C.) is then run in steps of −10% again allowing a maximum of four hours for equilibration or an asymptotic weight. Following desorption, an adsorption scan is performed from 0% RH to the initial 40% RH. The sample is then dried for one hour at 80° C. to obtain the dry weight.

Aqueous solubility determination is determined by high-performance liquid chromatography (HPLC). Five standards of the starting material are prepared (1.2-9.6 mg/ml), and a calibration curve is generated by analyzing the solutions using HPLC to obtain peak area counts. Approximately 10-100 mg of the salts and the starting material are suspended in 200-500 microliters of deionized water while stirring at ambient temperature overnight. Samples are then centrifuged and filtered. The supernatant liquid is removed and analyzed by HPLC to determine the solubility by comparing area counts with the calibration curve. The residual solids are isolated and analyzed by X-ray powder diffraction (XRPD) to evaluate potential form conversion.

Stability of the salt is evaluated using a seven day toluene slurry. Each salt (about 10-20 mg) is slurried in 250 microliters of toluene at ambient temperature for seven days in a 4 ml vial. After seven days, each sample is filtered and dried overnight at ambient temperature under vacuum. The samples are then analyzed by XRPD.

### Starting Material, Phenobarbital Free Form

Phenobarbital free form starting material in crystalline form is analyzed by XRPD. DSC analysis reveals an endothermic event at 175° C. attributed to the melt and no weight loss is observed by TGA below 200° C. Moisture sorption analysis indicates the material to be nonhygroscopic adsorbing 0.1 wt % water at 90% RH. Upon desorption, slight hysteresis is observed between 65-25% RH. XRPD analysis of the dried material following the moisture sorption experiment affords a diffraction pattern consistent with the initial phenobarbital starting material. Following the water slurry, the residual solids are analyzed by XRPD resulting in a unique pattern from the starting form. The aqueous solubility of this freeform is 1.1 mg/ml. Further analysis by DSC affords a minor endotherm at 167° C. which may be attributed to the melt of the new form followed by major endotherms at 174° C. and 176° C. which may be attributed to the melting of the recrystallized freeform.

### Example 1

### Preparation of Phenobarbital Potassium Salt

Preparation of phenobarbital potassium salt is achieved by dissolving phenobarbital and potassium hydroxide in isopropanol followed by removal of the isopropanol. The obtained material is analyzed by XRPD and is shown to have a different pattern than phenobarbital free form. The potassium salt exhibits a single endothermic event attributed to the melt at 324° C. by DSC and an 8.2% weight loss below 140° C. is observed by TGA indicative of a loss of water (the theoretical value of a monohydrate is 5.9%). ¹H-NMR analysis is consistent with the chemical structure of phenobarbital showing little to no residual isopropanol. Moisture sorption analysis indicates the material is moderately hygroscopic adsorbing a maximum o 10.8 wt % water at 90% RH. During desorption, the curve stabilizes from 80-5% RH suggesting a stable monohydrate. XRPD analysis of the material following the moisture sorption experiment affords a diffraction pattern consistent with the initial material. The residual solid isolated from the aqueous solubility is analyzed by XRPD and found to be unique compared to the initial material and consistent with the pattern obtained from a water slurry of phenobarbital free form suggesting potential dissociation if the initial salt form. The aqueous solubility is 88.6 mg/ml. XRPD analysis of the material following a seven day toluene slurry is consistent with the initial material.

### Example 2

### Preparation of Phenobarbital Betaine Salt

Preparation of phenobarbital betaine salt is achieved by dissolving phenobarbital and betaine in dimethylformamide followed by removal of the dimethylformamide. The obtained material is analyzed by XRPD and is shown to have a different pattern than phenobarbital free form. An endothermic event attributed to a loss of water and/or DMF is observed at approximately 71° C. by DSC which corresponds to a 1-2% weight loss by TGA. DSC endotherms are also observed at 197° C. and 260° C. which can be attributed to melting of the salt followed by decomposition. ¹H-NMR analysis shows a counterion to API ratio of approximately 1:1 suggesting a mono-betaine salt. Residual DMF (1.0 wt %) is observed by ¹H-NMR. Moisture sorption analysis indicates the material is hygroscopic and the adsorption curve shows a stable hemihydrate between 20-50% RH. Above 50% RH a sharp increase in water adsorped is observed to a maximum of 28.1 wt % at 90% RH. Upon desorption, a stable hemihydrate is observed between 45-5% RH. XRPD analysis of the material following the moisture sorption experiment affords a diffraction pattern consistent with the initial material. The residual solid isolated from the aqueous solubility is analyzed by XRPD and found to be unique compared to the initial material and consistent with the pattern obtained from a water slurry of phenobarbital free form suggesting potential dissociation if the initial salt form. The aqueous solubility is 1.9 mg/ml. DSC thermogram of the residual solid is consistent with the thermogram of the solids isolated from the freeform water slurry showing a minor endotherm at 166° C. followed by major endotherms at 174° C. and 176° C. ¹H-NMR analysis of the residual solid shows a counterion to API ratio of 0.1:1 suggesting salt dissociation. XRPD analysis of the material following a seven day toluene slurry is consistent with the initial material.

### Example 3

### Preparation of Phenobarbital Choline

Preparation of phenobarbital choline salt is achieved by dissolving phenobarbital and choline in isopropanol followed by removal of the isopropanol. The obtained material is analyzed by XRPD and is shown to have a different pattern than phenobarbital free form. DSC analysis reveals a minor endotherm is observed at 165° C. followed by major endotherms at 196° C. and 218° C. which can be attributed to melt followed by decomposition. A TGA weight loss of 6.6 wt % was observed and may be attributed to loss of water or IPA. ¹H-NMR analysis shows a counterion to API ratio of approximately 1.2:1 indicating a monocholine salt. An amount (1.2 wt %) of isopropanol is observed by ¹H-NMR. Moisture sorption analysis indicates the material is hygroscopic adsorbing 23 wt % water at 90% RH. Upon desorption, no hysteresis is observed. XRPD analysis of the material following the moisture sorption experiment affords a diffraction pattern consistent with the initial material. The aqueous solubility is 294.9 mg/ml. The solids from the water slurry are analyzed by ¹H-NMR indicating a counterion to API ratio of 1.2:1 showing no degradation suggesting the salt remained stable throughout the experiment. The residual solid isolated from the aqueous solubility and seven day toluene slurry experiments are analyzed by XRPD and found to be consistent with the initial material.

### Example 4

### Preparation of Phenobarbital Benzathine

Preparation of phenobarbital benzathine (N,N′-dibenzylethylenediamine) salt is achieved by dissolving phenobarbital and benzathine in tetrahydrofuran followed by removal of the tetrahydrofuran. The obtained material is analyzed by XRPD and is shown to have a different pattern than phenobarbital free form. DSC analysis reveals endothermic events at 81° C. and 307° C. and no weight loss was observed by TGA. ¹H-NMR analysis shows a counterion to API ratio of 1.4:1 indicating a monobenzathine salt. Moisture sorption analysis indicates the material is nonhygroscopic adsorbing 1.0 wt % water at 90% RH. XRPD analysis of the material following the moisture sorption experiment affords a diffraction pattern consistent with the initial material. The aqueous solubility is 2.9 mg/ml. The solids from the water slurry are analyzed by ¹H-NMR indicating a counterion to API ratio of 1.2:1 showing no significant degradation suggesting the salt remained stable throughout the experiment. The residual solid isolated from the aqueous solubility is analyzed by XRPD and found to be consistent with the initial material.

### Example 5

### Preparation of Phenobarbital Sodium Salt, Amorphous

Preparation of phenobarbital sodium salt is achieved by dissolving phenobarbital and sodium hydroxide in acetone followed by removal of the acetone. The obtained material is analyzed by XRPD and is shown to have a different pattern than phenobarbital free form. The sodium salt exhibits a single endothermic event attributed to the melt at 297° C. by DSC and a continuous weight loss is observed by TGA. ¹H-NMR analysis is consistent with the chemical structure of phenobarbital. Moisture sorption analysis indicates the material is hygroscopic adsorbing approximately 34 wt % water at 90% RH. Between 10-60% RH the sample adsorbed approximately 2 wt % water indicating a sesqui or dihydate. Karl Fischer analysis of the starting material shows approximately 10 wt % water which is comparable to a sesquihydate (9.0 wt %). Upon desorption, the curve stabilizes at approximately 12 wt % water indicating a stable dihydrate from 40-5% RH. XRPD analysis of the material following the moisture sorption experiment affords a diffraction pattern which shows differences in comparison to the diffraction pattern of the initial material which may be due to a change in hydration state following drying. Following water slurry and amorphous XRPD pattern is observed and the aqueous solubility of the amorphous material is 86.5 mg/ml. The residual solid isolated from the seven day toluene slurry are analyzed by XRPD and is amorphous.

### Example 6

### Solubility Analyses

The phenobarbital salts will be tested for intrinsic dissolution, dissolution rate, and equilibrium solubility.

Intrinsic dissolution will be determined using apparatus and procedures according to USP 31 <1087>. A selected phenobarbital salt will be compressed in a die to form a pellet, the pellet will then be fixed into a dissolution shaft which has a holder for the pellet. The pellet will then be exposed to a dissolution medium with constant surface area to eliminate the effect of particle size. The dissolution medium will be 250 mL or 500 mL of deionized water.

Dissolution rate will be determined by placing a selected phenobarbital salt powder into different media under sink conditions using a platform shaker. The sink conditions involves maintaining a volume of dissolution medium that is 5 to 10 times greater than the volume at the saturation point of the active agent contained in the active agent delivery system being tested. Generally, for high solubility and high permeability active agents, the sink condition is 3 times, for a low solubility active agents more than 5 times the volume is used to maintain the active agent in sink condition. Samples will be withdrawn at various time points. The dissolution will be carried out at ambient temperature. Characterization of active agents as high solubility, high permeability, low solubility, or low permeability can be found in the discussion of the Biopharmaceutics Classification System in the document Guidance for Industry Waiver of In Vivo Bioavailability and Bioequivalence Studies for Immediate-Release Solid Oral Dosage Forms Based on a Biopharmaceutics Classification System U.S. Food and Drug Administration Center for Drug Evaluation and Research (CDER) August 2000.

Equilibrium solubility will be determined by adding a selected phenobarbital salt powder into different media at an over-saturated condition. A Dissolution bath apparatus II (USP 31 <711> (paddle)). will be used and fitted with a fiber optic device that will read the UV absorbance every minute or at shorter time intervals.

In general, the media used for the dissolution rate and equilibrium solubility studies will be aqueous media over the pH range of 1 to 7.5 using a.) 0.1N HCl or simulated Gastric Fluid USP without enzymes for a pH of 1.2, b.) a pH 4.5 buffer, and c.) pH 6.8 buffer or simulated intestinal fluid USP without enzymes. A minimum of three replicate determinations of solubility in each pH condition will be made recommended. The concentration of phenobarbital in selected buffers or pH conditions will be determined using a stability indicating assay that will distinguish the active agent from its degradation products, and will also detect any degradation that may appear in the salt solution.

The terms “comprising”, “having”, “including”, and “containing” are to be construed as open-ended terms (i.e., meaning “including, but not limited to”). The terms “a” and “an” do not denote a limitation of quantity, but rather denote the presence of at least one of the referenced item. The term “or” means “and/or”. The endpoints of all ranges directed to the same component or property are inclusive and independently combinable.

Unless defined otherwise, technical and scientific terms used herein have the same meaning as is commonly understood by one of skill in the art to which this invention belongs.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound which is a crystalline salt of phenobarbital selected from the group consisting of phenobarbital potassium, phenobarbital benzathine, phenobarbital betaine, and corresponding anhydrous forms, hydrates, solvates, or polymorphic forms thereof.

2. The compound of claim 1, wherein the salt of phenobarbital is phenobarbital benzathine salt.

3. The compound of claim 1, wherein the salt of phenobarbital is phenobarbital betaine salt.

4. The compound of claim 1, wherein the salt of phenobarbital is phenobarbital potassium salt.

5. The compound of claim 1, wherein the salt of phenobarbital is phenobarbital potassium salt, monohydrate.

6. Amorphous phenobarbital sodium salt or a corresponding anhydrous form, hydrate, or solvate thereof

7. A method of preparing a crystalline salt of phenobarbital, comprising:
crystallizing or precipitating a phenobarbital salt from a solution of phenobarbital, a counterion source, and a solvent;
wherein the crystalline salt of phenobarbital is selected from the group consisting of phenobarbital potassium, phenobarbital benzathine, phenobarbital betaine, and corresponding anhydrous forms, hydrates, solvates, or polymorphic forms thereof.

8. The method of claim 7, comprising precipitating phenobarbital benzathine from a solution of phenobarbital, benzathine, and tetrahydrofuran by removal of tetrahydrofuran.

9. The method of claim 7, comprising
precipitating phenobarbital betaine from a solution of phenobarbital, betaine, and dimethylformamide by removal of dimethylformamide.

10. The method of claim 7, comprising
precipitating phenobarbital potassium from a solution of phenobarbital, potassium hydroxide, and isopropanol by removal of isopropanol.

11. A composition comprising:
a compound which is a crystalline salt of phenobarbital selected from the group consisting of phenobarbital potassium, phenobarbital benzathine, phenobarbital betaine, and corresponding anhydrous forms, hydrates, solvates, or polymorphic forms thereof; and
a pharmaceutically acceptable excipient.

12. The composition of claim 11, wherein the composition is an oral or parenteral formulation.

13. The composition of claim 11, wherein the composition is an intravenous, transdermal, subcutaneous, subcutaneous depot, or intramuscular formulation.

14. A method of treating, comprising:
administering to a patient in need of phenobarbital therapy the compound of claim 1.

15. A method of treating, comprising:
administering to a patient in need of phenobarbital therapy the composition of claim 11.

16. The method of claim 14, wherein the administering is to treat or control seizures, grand mal seizures, complex or simple partial seizures in adults and children, generalized tonic-clonic seizures, myclonic seizures and neonatal seizures, febrile seizures, status epilepticus; short-term treatment of anxiety; or short-term treatment of insomnia.

17. The method of claim 15, wherein the administering is to treat or control seizures, grand mal seizures, complex or simple partial seizures in adults and children, generalized tonic-clonic seizures, myclonic seizures and neonatal seizures, status epilepticus; short-term treatment of anxiety; or short-term treatment of insomnia.
